(19) United States
(12) Patent Application Publication
(10) Pub. No.: US 2009/0131966 A1
(43) Pub. Date: May 21, 2009

(54) Single-puncture lancing system

(76) Inventors: Mohammad Kheiri, Elkhart, IN (US); Weiping Zhong, Granger, IN (US); Tieming Ruan, Granger, IN (US)

Correspondence Address: NIXON PEABODY LLP, 161 N. CLARK STREET, 48TH FLOOR, CHICAGO, IL, 60601, US

(21) Appl. No.: 11/921,670

(22) PCT Filed: Jun. 29, 2006
(86) PCT No.: PCT/US2006/025307

§ 371 Date: Dec. 6, 2007

Related U.S. Application Data
(60) Provisional application No. 60/695,388, filed on Jun. 30, 2005.

(51) Int. Cl. A61B 5/151

(52) U.S. Cl. 606/182

(57) ABSTRACT

A lancing device (10) comprises a main housing (12), a moveable housing (14) and one or more contacting members (68a, 68b, 66a, 66b). The main housing forms an inner cavity (24) enclosing a portion of a lancing mechanism. The lancing mechanism includes a lancet holder (36) attached to a shaft (38) having an enlarged end (40). The main housing further encloses a drive spring (42) surrounding a portion of the shaft. The lancing mechanism is adapted to move between resting, cocking and puncture positions. The movable housing is adjacent the main housing and moves from a resting position to a cocking position. The moveable housing forms an inner cavity (26) enclosing a portion of the shaft of the lancing mechanism. The contacting member is enclosed within the lancing device. The contacting member has a damping pad (68a, 68b) and a stopper (66a, 66b) and engages a portion of the lancing mechanism when moved from the cocking position to the puncture position.

### FIELD OF THE INVENTION

The present invention relates generally to lancing devices and, more particularly, to a single-puncture lancing mechanism for a lancing device.

### BACKGROUND OF THE INVENTION

The quantitative determination of analytes in body fluids is of great importance in the diagnoses and maintenance of certain physiological abnormalities. For example, lactate, cholesterol and bilirubin should be monitored in certain individuals. In particular, determining glucose in body fluids is important to diabetic individuals who must frequently check their blood glucose levels to regulate the glucose intake in their diets.

One method of obtaining a body fluid sample, such as a whole blood sample, is to use a lancing device. The whole blood sample may then be used to determine the glucose concentration of an individual. Existing lancing devices use a lancet to pierce the tissue of the skin, allowing a blood sample to form on the skin's surface. Typically, lancing devices hold the lancet within them when the lancet is not in use, so as to shield the user from injury as well as to assist in preventing or inhibiting contamination.

The whole blood sample is often obtained by piercing the skin of a test subject. In addition to the pain and discomfort inherent with such a puncture, existing lancing devices may cause increased pain to many individuals by failing to properly dampen the lancet after initially piercing the skin. This may result in multiple punctures to the individual's skin, increasing the discomfort to the user. Alternatively, excessive damping can reduce the lancet's force and adversely effect the puncture depth, causing insufficient sample size and the need to lance again.

Additionally, the cost, complexity, effectiveness, and design of lancing devices are important to individual user's. Thus, it would be desirable to have a lancing device and method that address these issues.

### SUMMARY OF THE INVENTION

According to one embodiment of the present invention, a lancing device is disclosed. The lancing device comprises a main housing, a movable housing, and one or more contacting members. The main housing forms an inner cavity enclosing a portion of a lancing mechanism. The lancing mechanism includes a lancet holder attached to a shaft having an enlarged end opposite the lancet holder. The main housing further encloses a drive spring surrounding a portion of the shaft. The drive spring is located between the lancet holder and a portion of the main housing. The lancing mechanism is adapted to move between a resting position, a cocking position, and a puncture position. The movable housing is adjacent the main housing and is adapted to move from a resting position to a cocking position. The moveable housing forms an inner cavity enclosing a portion of the shaft of the lancing mechanism. The one or more contacting members are enclosed within the lancing device. Each of the one or more contacting members has a damping pad and a stopper. The one or more contacting members are adapted to engage a portion of the lancing mechanism as the lancing mechanism moves from the cocking position to the puncture position.

According to another embodiment of the present invention, a lancing device is disclosed. The lancing device comprises a main housing, a movable housing, and a plurality of angled contacting members. The main housing forms an inner cavity enclosing a portion of a lancing mechanism. The lancing mechanism includes a lancet holder attached to a shaft. The lancet holder includes a plurality of angled bumpers formed thereon. The main housing further encloses a drive spring surrounding a portion of the shaft. The drive spring is located between the lancet holder and a portion of the main housing. The lancing mechanism is adapted to move between a resting position, a cocking position, and a puncture position. The movable housing is adjacent the main housing. The movable housing is adapted to move from a resting position to a cocking position. The moveable housing forms an inner cavity enclosing a portion of the shaft of the lancing mechanism. The plurality of angled contacting members is enclosed within the inner cavity of the main housing. The plurality of angled contacting members is adapted to engage the plurality of angled bumpers as the lancing mechanism approaches the puncture position. The engagement between the plurality of angled contacting members and the plurality of angled bumpers hinders the movement of the lancing mechanism as the lancing mechanism moves from the cocking position to the puncture position.

According to yet another embodiment of the present invention, a lancing device is disclosed. The lancing device comprises a main housing, a movable housing, and a contacting member. The main housing forms an inner cavity enclosing a portion of a lancing mechanism. The lancing mechanism includes a lancet holder attached to a shaft and a bumper formed thereon. The main housing further encloses a drive spring surrounding a portion of the shaft. The drive spring is located between the lancet holder and a portion of the main housing. The lancing mechanism is adapted to move between a resting position, a cocking position, and a puncture position. The movable housing is adjacent the main housing and is adapted to move from a resting position to a cocking position. The moveable housing forms an inner cavity enclosing a portion of the shaft of the lancing mechanism. The contacting member extends from a portion of the main housing. The contacting member is adapted to engage the bumper formed on the lancet holder. The contacting member is adapted to dissipate a first force from the lancing mechanism as the lancing mechanism moves from the cocking position to the puncture position and a second force from the lancing mechanism as the lancing mechanism moves from the puncture position to the resting position. The second force is substantially greater than the first force.

The above summary of the present invention is not intended to represent each embodiment, or every aspect, of the present invention. Additional features and benefits of the present invention are apparent from the detailed description and figures set forth below.

### BRIEF DESCRIPTION OF THE DRAWINGS

FIG. 1a is a top exploded view of a lancing device, according to one embodiment of the present invention.

FIG. 1b is a bottom exploded view of the lancing device of FIG. 1a.

FIG. 2 is a side view of the lancing device of FIGS. 1a-b.

FIG. 3 is a partial, cross-sectional, top view of the lancing device of FIG. 2 in a resting position.

FIG. 4 is a partial, cross-sectional, top view of the lancing device of FIG. 2 in a cocking position.

FIG. 5 is a perspective view of a lancing mechanism, according to one embodiment of the present invention.

FIG. 6a is a schematic cross-sectional view of a lancing device having a plurality of contacting members incorporated therein, according to one embodiment of the present invention.

FIG. 6b is a schematic cross-sectional view of a lancing device having a plurality of contacting members incorporated therein, according to another embodiment of the present invention.

FIG. 6c is a schematic cross-sectional view of a lancing device having a plurality of contacting members incorporated therein, according to yet another embodiment of the present invention.

FIG. 7 is a schematic cross-sectional view of a lancing device having a cantilever-beam with an angled end portion as a contacting member, according to one embodiment of the present invention.

FIG. 8a is a schematic cross-sectional view of a lancing device—having a cantilever-beam as a contacting member—with a lancing mechanism in a cocked position, according to another embodiment of the present invention.

FIG. 8b is a schematic cross-sectional view of the lancing device of FIG. 8a as the lancing mechanism moves from the cocked position to a puncture position.

FIG. 8c is a schematic cross-sectional view of the lancing device of FIG. 8a with the lancing mechanism in the puncture position.

FIG. 8d is a schematic cross-sectional view of the lancing device of FIG. 8a with the lancing mechanism stopped by the cantilever beam at an intermediate position between the puncture position and a resting position.

FIG. 8e is a schematic cross-sectional view of the lancing device of FIG. 8a with the slider having been moved to disengage the cantilever beam from the lancing mechanism.

FIG. 9 is a schematic cross-sectional view of a lancing device having a plurality of contacting members incorporated therein, according to yet another embodiment of the present invention.

### DESCRIPTION OF ILLUSTRATED EMBODIMENTS

The present invention is directed to a lancet-release mechanism for incorporation into a stand-alone lancing device or into a lancing device that is incorporated into a meter or similar testing device. The lancing device is adapted to receive a lancet for use in drawing a body fluid from a test subject. The body fluid generally contains at least one analyte that may then be examined to determine its concentration in the body fluid sample.

Lancing devices and lancets may be used to produce a blood or body fluid sample from a test subject. This sample may then be analyzed with a meter and test strip, or similar devices, to determine the concentration of the analyte to be examined. Examples of the types of analytes that may be collected with a lancing device include glucose, lipid profiles (e.g., cholesterol, triglycerides, LDL and HDL), microalbumin, hemoglobin A_{1c}, fructose, lactate, or bilirubin.

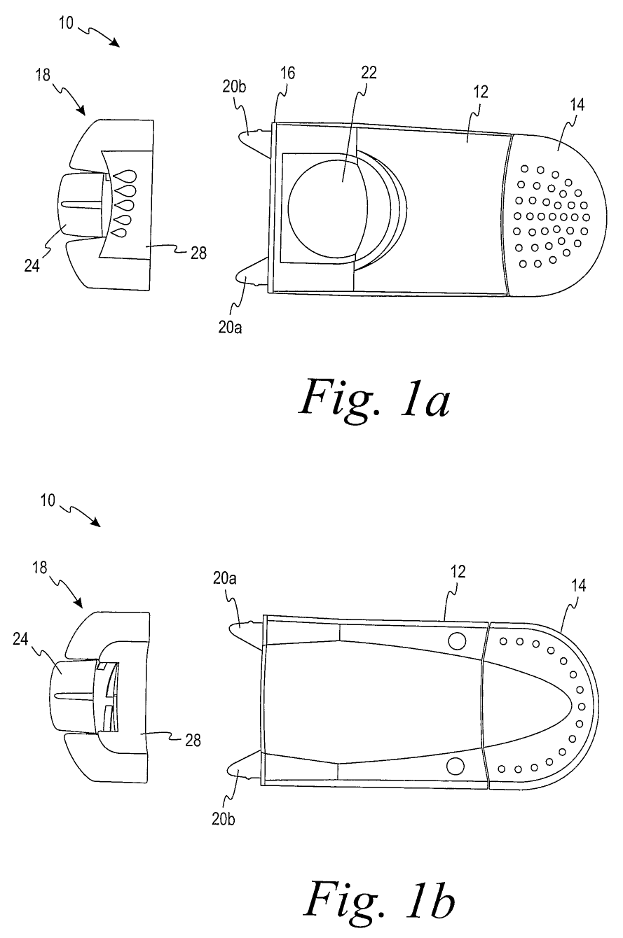

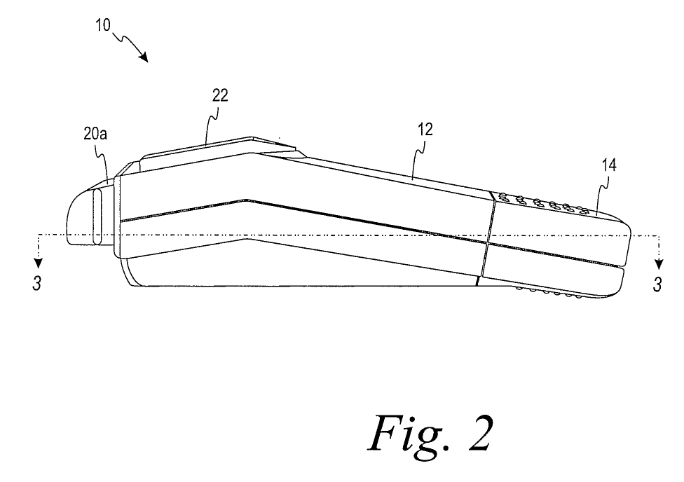

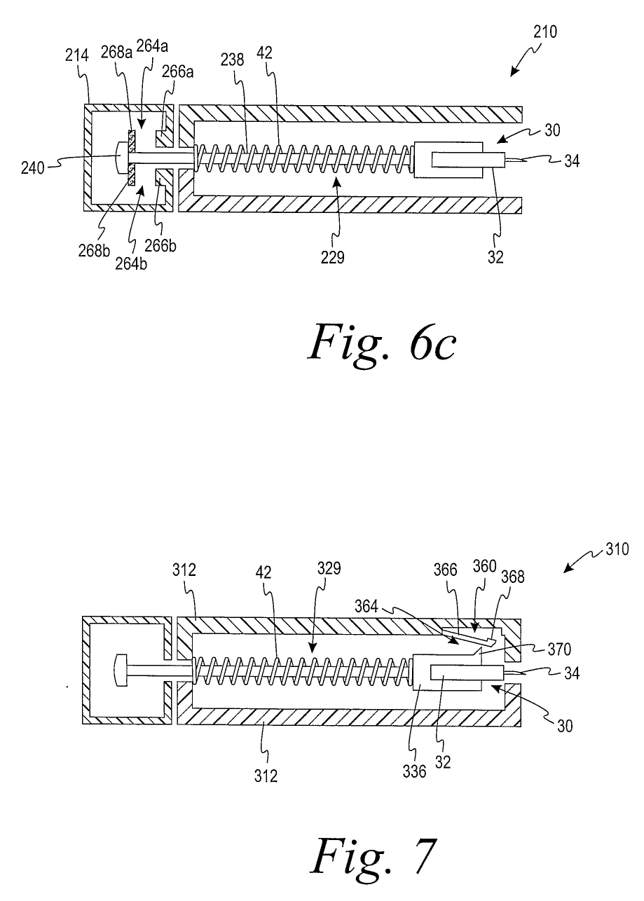

Turning now to the drawings and initially to FIGS. 1-2, a lancing device 10 for obtaining a fluid sample from a test subject is illustrated, according to one embodiment of the present invention. The lancing device 10 has a main housing 12 and a movable housing 14 that is movable relative to the main housing 12. The main housing 12 and the movable housing 14 each forms an inner cavity 24, 26 (FIG. 6a), respectively. An endcap support 16 is connected to the main housing 12 on the testing end of the lancing device 10. An endcap 18 may be removably attached to the endcap support 16. When attached, the endcap 18 is retained on the endcap support 16 by a pair of support arms 20a-b integrally formed with the endcap support 16.

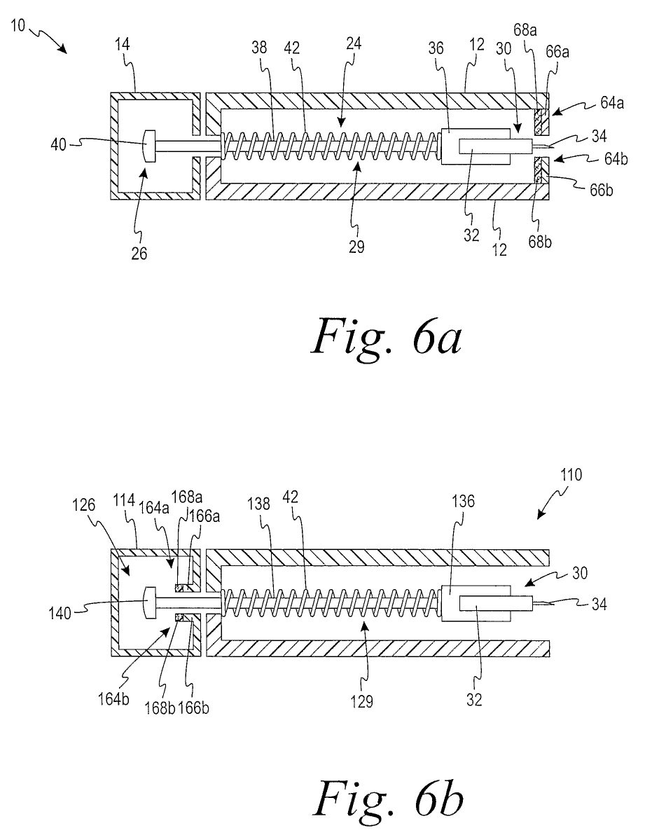

To use the lancing device 10, the movable housing 14 is pulled away from the main housing 12 to move an internal lancing mechanism 29 (as best illustrated in FIG. 5) to a cocked position, and then a pushbutton 22 is pushed to actuate the lancing mechanism 29 so that the sharp tip of a lancet is forced through an aperture (not shown) in the endcap 18. The lancing device 10 may be provided with a number of different endcaps 18, each having a different width, to facilitate the formation of skin punctures of various depths. Alternatively, the endcap 18 may include an adjustable dial 24 for allowing punctures of different depths to be performed utilizing a single endcap 18.

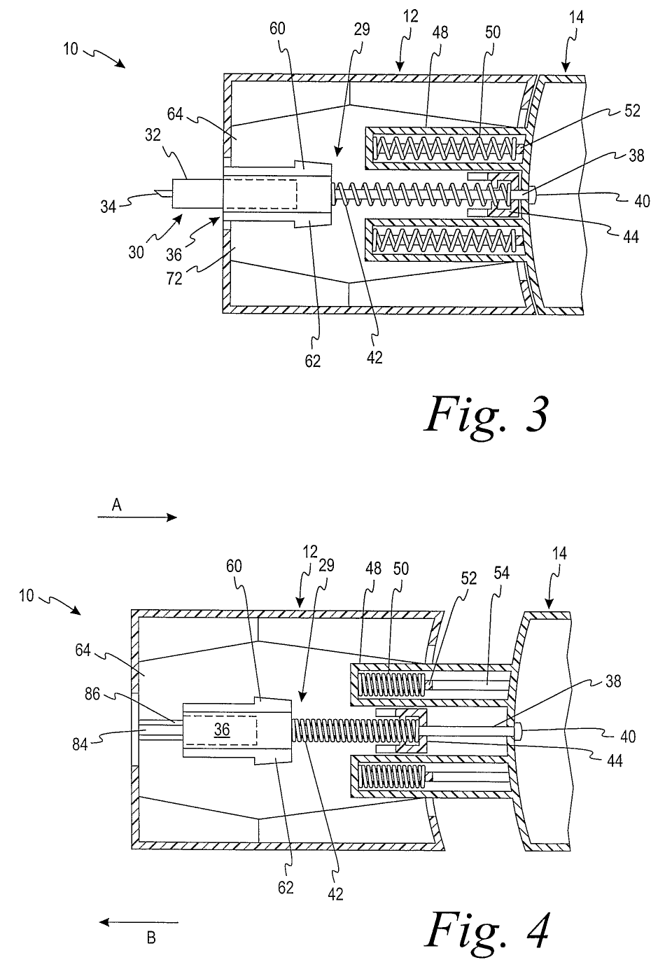

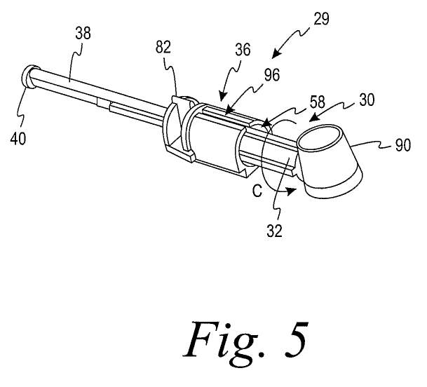

Turning now to FIG. 3, a cross-sectional view of a portion of the lancing device 10 is illustrated with the endcap 18 and endcap support 16 not shown. A lancet assembly 30 having a lancet body 32 and a lance 34 is received within an internal cylindrical aperture 58 (FIG. 5) formed in a generally cup-shaped lancet holder 36. The lancet holder 36 includes one or more angled stop member. In the illustrated embodiment (FIG. 3), the lancet holder 36 includes two angled stop members 60, 62 that are located on opposite sides of the lancet holder 36. The lancet holder 36 is connected to an elongated shaft 38 by being integrally formed therewith. The shaft 38 has an enlarged end 40 that is supported within the movable housing 14. A drive spring 42 is disposed around the shaft 38 between the lancet holder 36 and a spring stop 44 (see also FIG. 4) integrally formed with the main housing 12.

The movable housing 14 has a pair of elongate spring trays 48 integrally formed therewith. A return spring 50 is disposed within each of the spring trays 48, a first end of each return spring 50 being disposed against an internal surface of the spring tray 48 and a second end of each return spring 50 being disposed against a spring stop 52 integrally formed with the main housing 12. The spring stops 52 extend into the spring trays 48 through an elongate slot 54 (see FIG. 4) formed in the bottom portion of each tray 48.

FIG. 3 illustrates the interior of the lancing device 10 when the lancing device 10 is not in use. In this position, the lancet holder 36 is disposed in a resting position between a puncture position and a cocked position. FIG. 4 illustrates the interior of the lancing device 10 (the lancet assembly 30 is not shown) when the lancet holder 36 is in a cocked position in which the movable housing 14 has been pulled away from the main housing 12.

Referring to FIG. 4, to move the lancet holder 36 from its resting position to its cocked position, the movable housing 14 is pulled away from the main housing 12 in the direction of Arrow A. The movable housing 14 continues to be pulled—against the force of the drive spring 42—until the angled stop members 60, 62 formed on the lancet holder 36 move past (to the right of as illustrated in FIG. 4) at least one retaining arm (not shown) formed on the housing side of the push button 22. The at least one retaining arm extends into the housing 12. The at least one retaining arm is biased so that it makes contact with one or more of the angled stop members 60, 62 formed on the lancet holder 36. A spring mechanism—for example, an elastically-deformable foam material—may be disposed between the pushbutton 22 and a portion of the main housing 12 to bias the pushbutton 22 to its non-actuated position, wherein the at least one retaining arm is able to engage one or both of the angled stop members 60, 62.

When in that cocked position, movement of the lancet holder 36 in the direction of Arrow B due to the drive spring 42 is prevented because of the contact between the at lest one retaining arm and the angled stop members 60, 62. After the lancet holder 36 is placed in the cocked position, the user allows the return springs 50 to force the movable housing 14 back to its initial position adjacent the main housing 12.

The lancet holder 36 is guided between its resting and cocked positions by a guide rib 82 (FIG. 5) formed on the bottom portion of the lancet holder 36 that rides within a groove 84 formed between a pair of raised guide rails 86 formed in a bottom interior portion of the main housing 12.

To perform a puncture on a test subject's skin, the endcap 18 is attached to the lancing device 10. The lancet holder 36 may be in the cocked position at the time the endcap 18 is attached or may be cocked once the endcap 18 is in position. The endcap 18 is then placed firmly against the skin where the puncture is to be made, and the pushbutton 22 is depressed. Depressing the pushbutton 22 causes the at least one retaining aim (not shown) to release the angled stop members 60, 62 formed on the lancet holder 36. Thus, the lancet holder 36 is no longer prevented from moving in the direction of ArroW B by the contact of the at least one retaining arm with one or both of the angled stop members 60, 62.

Upon release of the lancet holder 36 as described above, the drive spring 42 will force the lancet holder 36 in the direction of Arrow B until the sharp point of the lance 34 (FIG. 3) passes through the aperture (not shown) in the endcap 18 to make the puncture. When the puncture is made, the drive spring 42 will be in a stretched position, and after the puncture is made the contraction of the drive spring 42 will draw the lancet holder 36 back towards its resting position shown in FIG. 3.

Turning now to FIG. 5, a perspective view of the lancing mechanism 29 is illustrated, according to one embodiment of the present invention. The lancet mechanism 29 includes the lancet holder 36 adapted to receive the lancet assembly 30. As illustrated in FIG. 5, the lancet assembly 30 is disposed within the generally cylindrical aperture formed in the lancet holder 36. The lancet assembly 30 is shown with a protective cap 90 that has a portion that is integrally formed with the lancet body 32 and which covers the sharp point of the lance 34. Prior to using the lancing device 10, the lancet body 32 of a new lancet assembly 30 is inserted into the cylindrical aperture disposed in the lancet holder 36, and then the protective cap 90 is twisted off of the lancet assembly 30, in the direction of the Arrow C shown in FIG. 5.

Turning now to FIGS. 6-9, a damping system that can be incorporated into the above-described lancing device will be illustrated, according to the various embodiments of the present invention. Such damping systems assist in preventing or inhibiting the drive spring 42—and its natural tendency to oscillate (due to its being elastically deformable)—from causing a second, unintended skin puncture to be made by the lancet assembly 30. The damping system includes one or more contacting member adapted to contact the lancing mechanism and prevent or inhibit multiple punctures of the test subject's skin.

Referring now to FIGS. 6a-6c, a damping system is illustrated that includes a plurality of damping pads located within the lancing device 10. In FIG. 6a, a plurality of stoppers 66a,b is formed within the inner cavity 24 of the main housing 12. The stoppers 66a,b may be integrally formed with the main housing 12 and may be made from the same material as the main housing 12. Alternatively, the stoppers 66a,b may be attached to the main housing 12 after being formed. A damping pad 68a,b is attached to a respective stopper 66a,b to form contacting members 64a,b. The damping pads 68a,b may be formed from a variety of energy-absorptive materials, such as, for example, foam, rubber, etc. The damping pads 68a,b are adapted to contact a portion of the lancing mechanism 29 and disperse a portion of the lancing mechanism's 29 kinetic energy within the damping pads 68a,b, thus, preventing or inhibiting the lancing mechanism 29 from repuncturing the test subject's skin.

As shown in FIG. 6a, the contacting members 64a,b are formed at the lancing end of the lancing device 10. The contacting members 64a,b are formed and positioned to allow the lancet assembly 30 to extend therethrough. As such, when the lancing mechanism 29 moves from the cocked position to the puncture position, the lance 34 and a portion of the lancet body 32 extend through the contacting members 64a,b and outside of the lancing device 10 to pierce the skin of test subject. The lancet holder 36 engages the contacting members 64a,b as the lancet holder 36 reaches the puncture position. By this point, the drive spring 42 has fully decompressed and imparted at least some of its energy to the lancet holder 36 to move the lancet holder 36 towards the puncture position.

The engagement between the contacting members 64a,b and the lancet holder 36 dissipates some of this energy from the lancet holder 36 by receiving some of the drive force with the dampening pads 68a,b. As the drive spring 42 returns the lancet holder 36 back towards the resting position, the drive spring 42 generally recompresses and decompresses. However, because some of the initial drive force has been dissipated by the contacting members 64a,b, the secondary decompression of the drive spring 42 is prevented or inhibited from causing the lance 34 of the lancet assembly 30 to re-extend from the lancing device 10, potentially repuncturing the skin of the test subject.

As illustrated in FIG. 6b, the plurality of contacting members 164a,b is located in an inner cavity 126 formed by the movable housing 114. As discussed above, the contacting members 164a,b each include a damping pad 168a,b attached to a respective stopper 166a,b. In one embodiment, the stoppers 166a,b are integrally formed on the movable housing 114, while in yet other embodiments the stoppers 166a,b are attached to the movable housing 114 after the movable housing 114 has been formed.

In the embodiment shown, an enlarged end 140 of a shaft 138 is adapted to more uniformly engage the contacting members 164a,b. It should be noted, however, that the enlarged end 40 (as best illustrated in FIGS. 5 and 6a) could alternatively be used in this embodiment.

The damping system of FIG. 6b is operationally similar to the damping system described with respect to FIG. 6a. When the lancing mechanism 129 moves from the cocked position to the puncture position, the lance 34 and a portion of the lancet body 32—received within the lancet holder 136—extend outside of the lancing device 110 to pierce the skin of test subject. The enlarged end 140 of the shaft 138 engages the contacting members 164a,b as the lancing device 129 is driven by the drive spring 42 to the puncture position. When the lancing device 110 is in the puncture position, the drive spring 42 is fully decompressed. The engagement between the contacting members 164a,b and the enlarged end 140 of the shaft 138 dissipates some of the energy imparted to the lancing mechanism 129 from the drive spring 42 by receiving some of the drive force with the dampening pads 168a,b.

As the drive spring 42 returns the lancet mechanism 129 back towards the resting position, the drive spring 42 generally recompresses and decompresses. However, because some of the initial drive force has been dissipated by the contacting members 164a,b, the secondary decompression of the drive spring 42 is prevented or inhibited from causing the lance 34 of the lancet assembly 30 to re-extend from the lancing device 110, potentially repuncturing the skin of the test subject.

FIG. 6c illustrates yet another configuration of a damping system having a plurality of contacting members 264a,b. In one embodiment, a plurality of stoppers 266a,b is integrally formed on the movable housing 214 on opposite sides of shaft 238, while in yet other embodiments the stoppers 266a,b are attached to the movable housing 214 after the movable housing 214 has been formed. A plurality of damping pads 268a,b is located on the enlarged end 240 of the shaft 238 opposite from one another. The damping pads 268a,b are adapted to engage the stoppers 266a,b as the lancing mechanism 229 reaches the puncture position. This engagement dissipates some of the force exerted on the lancing mechanism 229 from the drive spring 42. Because some of the initial drive force has been dissipated by the contacting members 264a,b, the secondary decompression of the drive spring 42 is prevented or inhibited from causing the lance 34 of the lancet assembly 30 to re-extend from the lancing device 210.

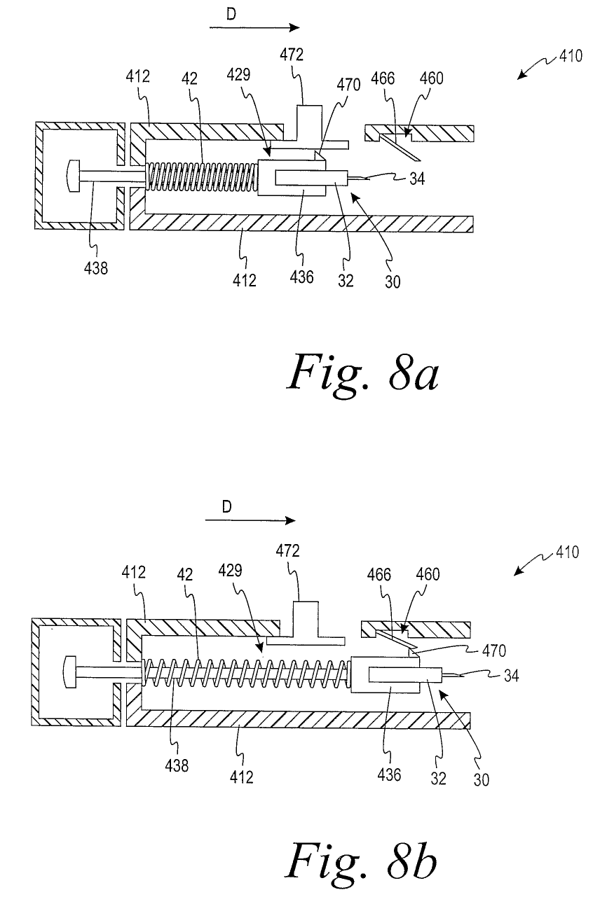

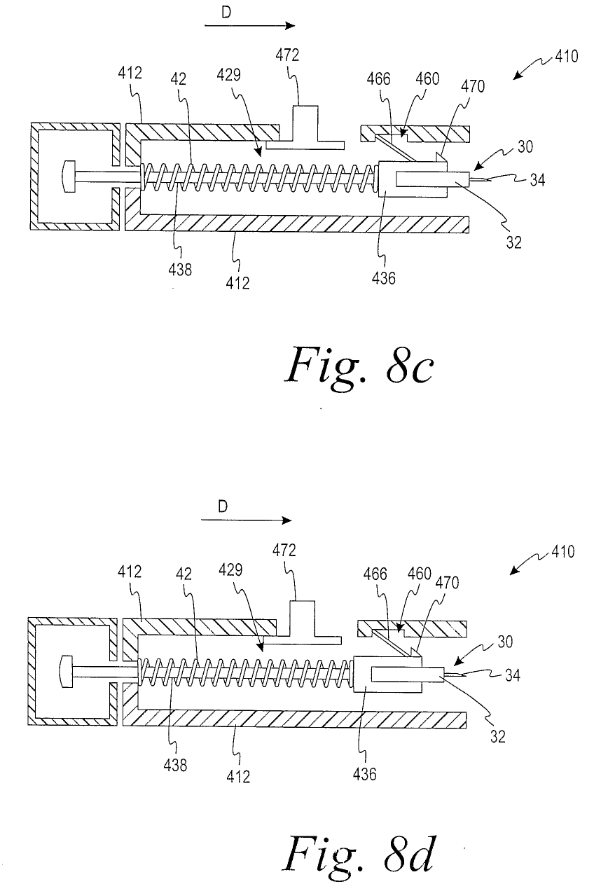

Turning now to FIGS. 7-8, a damping system is illustrated that comprises a cantilever beam adapted to contact a portion of a lancing mechanism and disperse a portion of the lancing mechanism's kinetic energy. As illustrated in FIG. 7, the damping system 364 includes a contacting member 366 having an angled end portion 368. The contacting member 366 is a cantilever beam integrally formed with a main housing 312 of a lancing device 310 during the molding process. The angled end portion 368 is adapted to engage a bumper 370 formed on a lancet holder 336.

A contacting member 366 is biased to engage the lancet holder 336 as a lancing mechanism 329 moves from the cocked position to the puncture position. The bias of the contacting member 366 also causes the contacting member 366 to engage the lancet holder 336 as the lancing mechanism 329 moves from the puncture position to the resting position. As the lancing mechanism 329 begins to move to the puncture position, the bumper 370 on the lancet holder 336 contacts the angled end portion 368 of the contacting member 366. As the lancing mechanism 329 continues to move to the puncture position, the contact of the bumper 370 with the angled end portion 368 causes the contacting member 366 to move away from the lancet holder 336 into a recess 360 formed in the main housing 312 of the lancing device 310. As the lancet holder 336 continues toward the puncture position, the bumper 370 disengages the contacting member 366 and the bias causes the contacting member 366 to move out of the recess 360 and return to its original position.

After reaching the puncture position, the drive spring 42 causes the lancing mechanism 329 to return to the resting position. As the lancing mechanism 329 moves towards the resting position, the bumper 370 again engages the contacting member 366. However, due to the positioning of the contacting member 366—and the angle of interaction between the angled end portion 368 and the bumper 370—a greater amount of force is necessary to move the contacting member 366 into the recess 360 as the lancing mechanism 329 moves to the resting position than was required as the lancing mechanism 329 moved to the puncture position. Thus, some of the energy imparted from the drive spring 42 to the lancing mechanism 329 is dissipated, which assists in preventing or inhibiting a second, unintended puncture of the test subject's skin.

It should be noted that in the illustrated embodiment, the angled end portion 368 and the bumper 370 are adapted to allow the lancing mechanism 329 to return to the resting position after having reached the puncture position. Alternatively, as illustrated in FIGS. 8a-e, a contacting member 466 may be adapted to engage a lancing mechanism 429 and prevent or inhibit the lancing mechanism 429 from returning to the resting position without further user interaction.

In FIG. 8a, the lancing mechanism 429 of a lancing device 410 is in the cocked position with the contacting member 466 biased away from a main housing 412 in the direction of the lancing mechanism 429. As illustrated in FIG. 8b, as the lancing mechanism 429 moves from the cocked position to the puncture position, a bumper 470 engages the contacting member 466 and moves the contacting member 466 towards a recess 460. The bumper 470 is angled so as to reduce the force necessary to move the contacting member 466. As illustrated in FIG. 8c, as the lancing mechanism 429 nears the puncture position, the bumper 470 disengages the contacting member 366 and the bias of the contacting member 366 causes the contacting member 366 to move away from the recess 460 and back to its resting position.

As the lancing mechanism 429 moves from the puncture position to the resting position, the contacting member 466 engages the bumper 470 of the lancet holder 436, as illustrated in FIG. 8d. This engagement causes the lancing mechanism 429 to stop prior to reaching the resting position. In this position the drive spring 42 is still slightly stretched and keeps the bumper 470 in engagement with the contacting member 466. To return the lancing mechanism 429 to the resting position, a user moves a slider 472 in the direction of Arrow D from a first position, shown in FIGS. 8a-d, to a second position shown in FIG. 8e. As the slider 472 is moved to the second position, the slider 472 engages the contacting member 466 and disengages the contacting member 466 from the bumper 470. Once the contacting member 466 disengages the bumper 470, the drive spring 42 compresses slightly and returns the lancing mechanism 429 to its resting position.

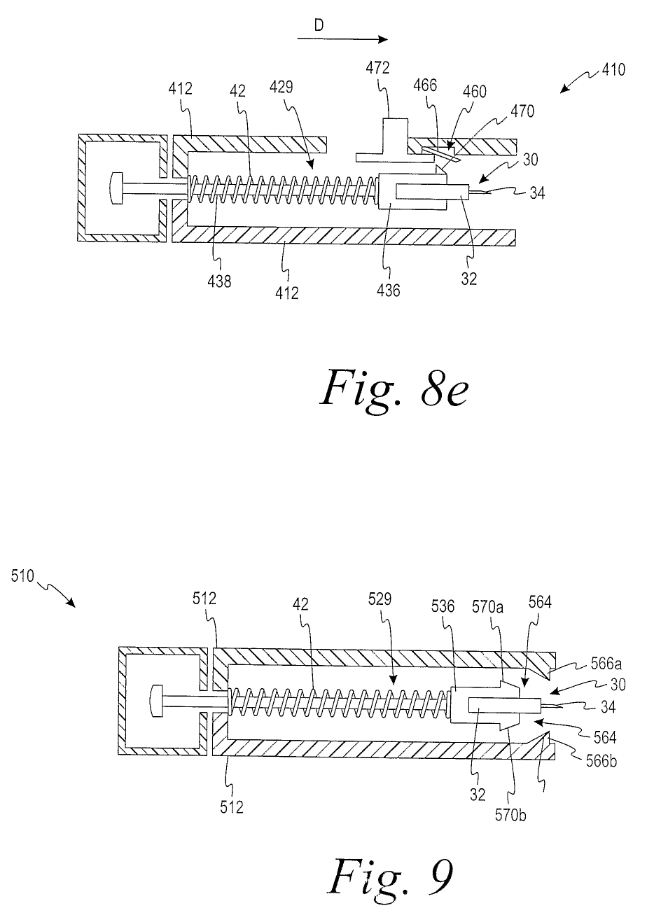

Turning now to FIG. 9, a damping system 564 adapted to be incorporated into a lancing device 510 is illustrated, according to yet another embodiment of the present invention. The damping system 564 includes a plurality of contacting members formed on the main housing 512. In the illustrated embodiment, two contacting members 566a,b are formed on the main housing 512 opposite each other. Each of the contacting members 566a,b is adapted to engage a respective bumper 570a,b formed on the lancet holder 536 as the lancing mechanism 529 moves from the cocked position to the puncture position. The contact between the bumpers 570a,b and the contacting members 566a,b is non-elastic and their engagement generates complex frictional forces, such as Coulombic friction and viscous friction. This dissipates some of the energy imparted to the lancing mechanism 529 by the drive spring 42 and assists in damping the lancing mechanism 529 to assist in preventing or inhibiting the lancet assembly from re-puncturing a test subject's skin.

The contacting members 566a,b and bumpers 570a,b may also assist in generating a consistent puncture depth by halting the forward movement of the lancing mechanism 529 at the puncture position after the lancing mechanism 529 has been fired. In the illustrated embodiment, the contacting members 566a,b and bumpers 570a,b are both triangular in shape. It is contemplated, however, that the contacting members 566a,b and/or bumpers 570a,b may be any of a variety of shapes and may be the same shape or different shapes. For example, the contacting members and/or bumpers may be circular, oblong, elliptical, trapezoidal, etc.

### Alternative Embodiment A

A lancing device comprising:

a main housing forming an inner cavity enclosing a portion of a lancing mechanism, the lancing mechanism including a lancet holder attached to a shaft having an enlarged end opposite the lancet holder, the main housing further enclosing a drive spring surrounding a portion of the shaft, the drive spring being located between the lancet holder and a portion of the main housing, the lancing mechanism being adapted to move between a resting position, a cocking position, and a puncture position;

a movable housing adjacent the main housing, the movable housing being adapted to move from a resting position to a cocking position, the moveable housing forming an inner cavity enclosing a portion of the shaft of the lancing mechanism; and

one or more contacting members enclosed within the lancing device, each of the one or more contacting members having a damping pad and a stopper, the one or more contacting members being adapted to engage a portion of the lancing mechanism as the lancing mechanism moves from the cocking position to the puncture position.

### Alternative Embodiment B

The lancing device of Alternative Embodiment A wherein the damping pad and the stopper are attached.

### Alternative Embodiment C

The lancing device of Alternative Embodiment A wherein the one or more contacting members are enclosed in the inner cavity of the main housing, the one or more contacting members being adapted to engage the lancet holder.

### Alternative Embodiment D

The lancing device of Alternative Embodiment A wherein the one or more contacting members are enclosed in the inner cavity of the movable housing, the one or more contacting members being adapted to engage the enlarged end of the shaft.

### Alternative Embodiment E

The lancing device of Alternative Embodiment D wherein the stopper of the one or more contacting members is formed as part of the movable housing.

### Alternative Embodiment F

The lancing device of Alternative Embodiment E wherein the damping pad of the one or more contacting members is attached to the stopper formed as part of the movable housing.

### Alternative Embodiment G

The lancing device of Alternative Embodiment E wherein the damping pad of the one or more contacting members is attached to the enlarged end of the shaft.

### Alternative Embodiment H

The lancing device of Alternative Embodiment A wherein the damping pad is formed from foam.

### Alternative Embodiment I

The lancing device of Alternative Embodiment A the damping pad is formed from rubber.

### Alternative Embodiment J

A lancing device comprising:

a main housing forming an inner cavity enclosing a portion of a lancing mechanism, the lancing mechanism including a lancet holder attached to a shaft, the lancet holder including a plurality of angled bumpers formed thereon, the main housing further enclosing a drive spring surrounding a portion of the shaft, the drive spring being located between the lancet holder and a portion of the main housing, the lancing mechanism being adapted to move between a resting position, a cocking position, and a puncture position;

a movable housing adjacent the main housing, the movable housing being adapted to move from a resting position to a cocking position, the moveable housing forming an inner cavity enclosing a portion of the shaft of the lancing mechanism; and

a plurality of angled contacting members enclosed within the inner cavity of the main housing, the plurality of angled contacting members being adapted to engage the plurality of angled bumpers as the lancing mechanism approaches the puncture position, the engagement between the plurality of angled contacting members and the plurality of angled bumpers hindering the movement of the lancing mechanism as the lancing mechanism moves from the cocking position to the puncture position.

### Alternative Embodiment K

The lancing device of Alternative Embodiment J wherein the plurality of contacting members and the plurality of angled bumpers are triangular shaped.

### Alternative Embodiment L

The lancing device of Alternative Embodiment J wherein the plurality of contacting members and the plurality of angled bumpers are circular shaped.

### Alternative Embodiment M

The lancing device of Alternative Embodiment J wherein the plurality of angled bumpers is circular shaped and the plurality of contacting members are inverted-circular shapes.

### Alternative Embodiment N

A lancing device comprising:

a main housing forming an inner cavity enclosing a portion of a lancing mechanism, the lancing mechanism including a lancet holder attached to a shaft, the lancet holder including a bumper formed thereon, the main housing further enclosing a drive spring surrounding a portion of the shaft, the drive spring being located between the lancet holder and a portion of the main housing, the lancing mechanism being adapted to move between a resting position, a cocking position, and a puncture position;

a movable housing adjacent the main housing, the movable housing being adapted to move from a resting position to a cocking position, the moveable housing forming an inner cavity enclosing a portion of the shaft of the lancing mechanism; and

a contacting member extending from a portion of the main housing, the contacting member being adapted to engage the bumper formed on the lancet holder, the contacting member being adapted to dissipate a first force from the lancing mechanism as the lancing mechanism moves from the cocking position to the puncture position, the contacting member being further adapted to dissipate a second force from the lancing mechanism as the lancing mechanism moves from the puncture position to the resting position, the second force being substantially greater than the first force.

### Alternative Embodiment O

The lancing device of Alternative Embodiment N wherein the contacting member is a cantilever beam.

### Alternative Embodiment P

The lancing device of Alternative Embodiment O, wherein the cantilever beam has an angled end portion located opposite the main housing, the angled end portion being adapted to engage the bumper formed on the lancet holder.

### Alternative Embodiment Q

The lancing device of Alternative Embodiment O wherein the lancing mechanism is stopped by the cantilever beam prior to reaching the resting position.

### Alternative Embodiment R

The lancing device of Alternative Embodiment Q further comprising a slider adapted to engage the cantilever beam and disengage the cantilever beam from the lancing mechanism.

### Alternative Embodiment S

The lancing device of Alternative Embodiment N wherein a recess is formed in a portion of the main housing, the recess being adapted to receive a portion of the cantilever beam therein.

While the invention is susceptible to various modifications and alternative forms, specific embodiments and methods thereof have been shown by way of example in the drawings and are described in detail herein. It should be understood, however, that it is not intended to limit the invention to the particular forms or methods disclosed, but, to the contrary, the intention is to cover all modifications, equivalents and alternatives falling within the spirit and scope of the invention as defined by the appended claims.

1. A lancing device comprising:
a main housing forming an inner cavity enclosing a portion of a lancing mechanism, the lancing mechanism including a lancet holder attached to a shaft having an enlarged end opposite the lancet holder, the main housing further enclosing a drive spring surrounding a portion of the shaft, the drive spring being located between the lancet holder and a portion of the main housing, the lancing mechanism being adapted to move between a resting position, a cocking position, and a puncture position;
a movable housing adjacent the main housing, the movable housing being adapted to move from a resting position to a cocking position, the moveable housing forming an inner cavity enclosing a portion of the shaft of the lancing mechanism; and
one or more contacting members enclosed within the lancing device, each of the one or more contacting members having a damping pad and a stopper, the one or more contacting members being adapted to engage a portion of the lancing mechanism as the lancing mechanism moves from the cocking position to the puncture position.

2. The lancing device of claim 1, wherein the damping pad and the stopper are attached.

3. The lancing device of claim 1, wherein the one or more contacting members are enclosed in the inner cavity of the main housing, the one or more contacting members being adapted to engage the lancet holder.

4. The lancing device of claim 1, wherein the one or more contacting members are enclosed in the inner cavity of the movable housing, the one or more contacting members being adapted to engage the enlarged end of the shaft.

5. The lancing device of claim 4, wherein the stopper of the one or more contacting members is formed as part of the movable housing.

6. The lancing device of claim 5, wherein the damping pad of the one or more contacting members is attached to the stopper formed as part of the movable housing.

7. The lancing device of claim 5, wherein the damping pad of the one or more contacting members is attached to the enlarged end of the shaft.

8. The lancing device of claim 1, wherein the damping pad is formed from foam.

9. The lancing device of claim 1, wherein the damping pad is formed from rubber.

10. A lancing device comprising:
a main housing forming an inner cavity enclosing a portion of a lancing mechanism, the lancing mechanism including a lancet holder attached to a shaft, the lancet holder including a plurality of angled bumpers formed thereon, the main housing further enclosing a drive spring surrounding a portion of the shaft, the drive spring being located between the lancet holder and a portion of the main housing, the lancing mechanism being adapted to move between a resting position, a cocking position, and a puncture position;
a movable housing adjacent the main housing, the movable housing being adapted to move from a resting position to a cocking position, the moveable housing forming an inner cavity enclosing a portion of the shaft of the lancing mechanism; and
a plurality of angled contacting members enclosed within the inner cavity of the main housing, the plurality of angled contacting members being adapted to engage the plurality of angled bumpers as the lancing mechanism approaches the puncture position, the engagement between the plurality of angled contacting members and the plurality of angled bumpers hindering the movement of the lancing mechanism as the lancing mechanism moves from the cocking position to the puncture position.

11. The lancing device of claim 10, wherein the plurality of contacting members and the plurality of angled bumpers are triangular shaped.

12. The lancing device of claim 10, wherein the plurality of contacting members and the plurality of angled bumpers are circular shaped.

13. The lancing device of claim 10, wherein the plurality of angled bumpers is circular shaped and the plurality of contacting members are inverted-circular shapes.

14. A lancing device comprising:
a main housing forming an inner cavity enclosing a portion of a lancing mechanism, the lancing mechanism including a lancet holder attached to a shaft, the lancet holder including a bumper formed thereon, the main housing further enclosing a drive spring surrounding a portion of the shaft, the drive spring being located between the lancet holder and a portion of the main housing, the lancing mechanism being adapted to move between a resting position, a cocking position, and a puncture position;
a movable housing adjacent the main housing, the movable housing being adapted to move from a resting position to a cocking position, the moveable housing forming an inner cavity enclosing a portion of the shaft of the lancing mechanism; and
a contacting member extending from a portion of the main housing, the contacting member being adapted to engage the bumper formed on the lancet holder, the contacting member being adapted to dissipate a first force from the lancing mechanism as the lancing mechanism moves from the cocking position to the puncture position, the contacting member being further adapted to dissipate a second force from the lancing mechanism as the lancing mechanism moves from the puncture position to the resting position, the second force being substantially greater than the first force.

15. The lancing device of claim 14, wherein the contacting member is a cantilever beam.

16. The lancing device of claim 15, wherein the cantilever beam has an angled end portion located opposite the main housing, the angled end portion being adapted to engage the bumper formed on the lancet holder.

17. The lancing device of claim 15, wherein the lancing mechanism is stopped by the cantilever beam prior to reaching the resting position.

18. The lancing device of claim 17, further comprising a slider adapted to engage the cantilever beam and disengage the cantilever beam from the lancing mechanism.

19. The lancing device of claim 14, wherein a recess is formed in a portion of the main housing, the recess being adapted to receive a portion of the cantilever beam therein.
